(19) United States
(12) Patent Application Publication
(10) Pub. No.: US 2023/0218255 A1
(43) Pub. Date: Jul. 13, 2023

(54) IMAGING OF MYELOMA BONE LESIONS

(71) Applicant: Blue Earth Diagnostics Limited, Oxford (GB)

(72) Inventor: Eugene TEOH, Oxford (GB)

(73) Assignee: Blue Earth Diagnostics Limited, Oxford (GB)

(21) Appl. No.: 18/151,204

(22) Filed: Jan. 6, 2023

Related U.S. Application Data
(60) Provisional application No. 63/297,374, filed on Jan. 7, 2022.

(51) Int. Cl. A61B 6/00; A61B 6/03; A61K 51/04

(52) U.S. Cl. CPC A61B 6/505; A61B 6/037; A61B 6/4417; A61K 51/0406

(57) ABSTRACT

The present disclosure relates to methods of administering [¹⁸F]-FACBC for imaging, diagnosing, and/or monitoring of myeloma bone lesions.

CROSS REFERENCE TO RELATED APPLICATION(S)

This application claims priority to U.S. Provisional Application No. 63/297,374, filed on Jan. 7, 2022, which is incorporated by reference herein in its entirety.

### TECHNICAL FIELD

The present disclosure relates to methods of administering [¹⁸F]-FACBC. The present invention also relates to use of [¹⁸F]-FACBC in methods for imaging and diagnosing myeloma bone lesions.

### BACKGROUND

Fluciclovine (¹⁸F), also known as [¹⁸F]-FACBC, FACBC, or anti-1-amino-3-¹⁸F-fluorocyclobutane-1-carboxylic acid, is a synthetic amino acid imaging agent which is taken up by amino acid transporters, and is used for positron emission tomography (PET). PET is uniquely suited to evaluate metabolic activity in human tissue for diagnostic imaging purposes. [¹⁸F]-fluoro-2-deoxy-glucose (FDG) is a PET imaging agent for the detection and localisation of many forms of cancer. However, FDG-PET has been found to have less sensitivity and/or specificity for assessment of some types of cancer, for example, myeloma.

The detection of focal bone lesions by imaging in suspected myeloma directly informs the patient's final diagnosis and subsequent management. Thus there is a need for a method of imaging which allows for reproducible, reliable imaging for detection and monitoring of myeloma in the bones.

### SUMMARY

The present disclosure relates to methods of diagnosing myeloma bone lesions in a subject by administering [¹⁸F]-FACBC for improved PET imaging and more reliable diagnosis of cancer or the recurrence thereof. In particular the present disclosure relates to methods of administering [¹⁸F]-FACBC for improved PET imaging and more reliable diagnosis of myeloma bone lesions and recurrence thereof. The present disclosure further relates to a method of diagnosing myeloma bone lesions and the recurrence thereof, using the PET imaging agent [¹⁸F]-FACBC. Comparison of the uptake of [¹⁸F]-FACBC uptake in a potential lesion against [¹⁸F]-FACBC uptake in the marrow may allow a more accurate diagnosis of the lesion.

In an aspect of the present disclosure, there is provided a method of diagnosing myeloma bone lesions in a subject comprising:
a) administering a detectable amount of [¹⁸F]-FACBC to the subject;
b) acquiring one or more positron emission tomography (PET) scan images of the subject for a region (the region including one or more areas of interest) of the subject that includes a potential myeloma bone lesion; and
c) comparing [¹⁸F]-FACBC uptake in the potential myeloma bone lesion against [¹⁸F]-FACBC uptake in the surrounding bone marrow of the region; wherein lesion [¹⁸F]-FACBC uptake is higher than the surrounding bone marrow [¹⁸F]-FACBC uptake.

In an aspect of the present disclosure, there is provided a method of using [¹⁸F]-FACBC, comprising the steps of:
a) administering a detectable amount of [¹⁸F]-FACBC to a subject;
b) acquiring one or more positron emission tomography (PET) scan images of the subject for a region of the subject that includes a potential myeloma bone lesion;
c) comparing [¹⁸F]-FACBC uptake in the potential myeloma bone lesion against [¹⁸F]-FACBC uptake in the surrounding bone marrow of the region; and
d) determining whether the potential lesion is a myeloma bone lesion based on the analysis of step c).

In an exemplary method, lesion [¹⁸F]-FACBC uptake is visually compared to [¹⁸F]-FACBC activity in the bone marrow. [¹⁸F]-FACBC uptake higher than the bone marrow is considered as suspicious for myeloma. [¹⁸F]-FACBC uptake similar or less than the bone marrow is considered as non-suspicious for myeloma.

In another aspect of the present disclosure there is provided a kit for imaging, diagnosing and/or monitoring myeloma, comprising: a) [¹⁸F]-FACBC tracer; and b) administration instructions according to the methods disclosed herein.

In an aspect of the present disclosure there is provided the acquisition of images using a PET/MRI or PET/CT scanner. The simultaneous or consecutive acquisition of images on a PET/MRI or PET/CT scanner may offer improved diagnostic accuracy over the generation of images on separate instruments. The conjoint use with CT or MRI may improve the localisation of lesions, particularly where the lesions are small (e.g., largest cross-section <1 cm), for example in the case of small myeloma bone lesions. The method of administration can be the method described herein.

In an aspect of the present disclosure, there is a method of using [¹⁸F]-FACBC, comprising the steps of:
a) administering a detectable amount of [¹⁸F]-FACBC to a subject; and
b) acquiring one or more positron emission tomography (PET) scan images of the subject for a region of the subject that includes a potential myeloma bone lesion;
wherein [¹⁸F]-FACBC uptake in the potential myeloma bone lesion is compared against [¹⁸F]-FACBC uptake in the surrounding bone marrow to determine whether the potential lesion is a myeloma bone lesion;
wherein the subject is positioned in a supine position with arms by the side before the PET scan begins, the scan starts from the subject's vertex and proceeds to the lowest extremity of the subject, and the scan begins 3 to 5 minutes after the [¹⁸F]-FACBC administration and continues for a total scan time of up to 30 minutes;
wherein the subject has been advised to:
i) consume no food or calorie-containing drink for at least 4 hours prior to administration of the [¹⁸F]-FACBC, with the exception of clear water and medications; and
ii) avoid significant exercise for at least one day prior to the PET scan; and
wherein [¹⁸F]-FACBC uptake by the potential myeloma bone lesion higher than the surrounding bone marrow [¹⁸F]-FACBC uptake is indicative of the presence of myeloma.

In an aspect of the present disclosure, there is a method of using [¹⁸F]-FACBC, comprising the steps of:
obtaining one or more positron emission tomography (PET) scan images of a region of a subject that includes a potential myeloma bone lesion, wherein the one or more PET scan images are acquired while the subject is positioned in a supine position with arms by the side, the scan starts from the subject's vertex and proceeds to the lowest extremity of the subject, and the scan begins 3 to 5 minutes after administration of [¹⁸F]-FACBC to the subject and continues for a total scan time of up to 30 minutes;
analysing the one or more PET scan images by comparing [¹⁸F]-FACBC uptake in the potential myeloma bone lesion against [¹⁸F]-FACBC uptake in the surrounding bone marrow; and determining a status of the potential myeloma bone lesion based on the analysis, wherein [¹⁸F]-FACBC uptake by the potential myeloma bone lesion higher than the surrounding bone marrow [¹⁸F]-FACBC uptake is indicative of the presence of myeloma.

Such methods may further comprise prescribing a treatment regime for the subject based on the determined status of the potential myeloma bone lesion.

### BRIEF DESCRIPTION OF DRAWINGS

FIG. 1: Coronal [¹⁸F]-FACBC PET (left) and fused PET/CT (right) images of the lumbar vertebrae. The arrow in the right image indicates an area diagnosed as a lesion, having [¹⁸F]-FACBC uptake higher than the surrounding bone marrow (indicated by squares). The PET scan acquisition was started 5 minutes after administration of 319 MBq of [¹⁸F]-FACBC.

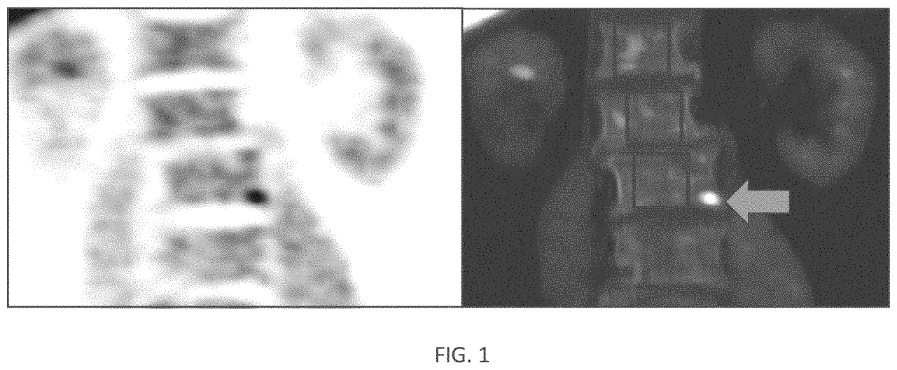

### DETAILED DESCRIPTION

The term “about” refers to being nearly the same as a referenced number or value and generally should be understood to encompass ±5% of a specified amount or value.

The present inventors have established a protocol which allows for more reliable imaging of bone lesions, e.g., allowing the data from PET scan images to be analysed and compared in order to more accurately diagnose and/or monitor myeloma bone lesions and areas suspected of being myeloma bone lesions. Comparison of the uptake of [¹⁸F]-FACBC uptake in a potential lesion against [¹⁸F]-FACBC uptake in the surrounding bone marrow may allow a more accurate diagnosis of the lesion.

Disclosed are methods of detecting lesions in a subject (e.g., a patient), comprising administering [¹⁸F]-FACBC to the subject and imaging the subject on a PET/MRI or PET/CT scanner to obtain conjoint PET and MRI images and/or PET and CT images. The lesions may be myeloma bone lesions. The administration and/or PET imaging methods may be as described below.

In an aspect of the present disclosure, there is provided a method of diagnosing myeloma bone lesions in a subject comprising the steps of:
(a) administering a detectable amount of [¹⁸F]-FACBC to the subject;
(b) acquiring one or more positron emission tomography (PET) scan images of the subject for a region of the subject that includes a potential myeloma bone lesion; and
(c) comparing [¹⁸F]-FACBC uptake in the potential lesion against [¹⁸F]-FACBC uptake in the bone marrow of the region of the subject; wherein lesion [¹⁸F]-FACBC uptake which is higher than bone marrow [¹⁸F]-FACBC uptake is indicative of the presence of myeloma bone lesions.

In step a), [¹⁸F]-FACBC may be injected as a bolus intravenous injection. The injection can be followed by a saline flush, e.g., a saline flush of about 10 mL or less.

In step a), [¹⁸F]-FACBC may be injected as a bolus intravenous injection. The injection can be followed by a saline flush, e.g., a saline flush of about 10 mL or less.

Amino acids are important nutrients for lesion growth. After injection, lesion cells are understood to uptake [¹⁸F]-FACBC, and the cells which have taken up the tracer can be subsequently visualised, e.g., via PET imaging. Acquisition of a PET scan image can start 3 to 5 minutes after the end of the injection, for example 4 minutes after the end of the injection. In some examples, acquisition can start 3, 4 or 5 minutes after the end of the injection. [¹⁸F]⁻ FACBC may be taken up by lesion cells relatively quickly compared to other PET radiotracers with different uptake mechanisms. For example, In FDG-PET imaging, acquisition usually starts at least 45 minutes after injection.

A “detectable amount” of [¹⁸F]-FACBC refers to a dosage of [¹⁸F]-FACBC which is taken up by lesion cells allowing those cells to be detected by PET imaging. In some examples, the dosage of [¹⁸F]-FACBC is 370±20% MBq, e.g., 370 MBq (+/−10%) or 370 MBq (+/−5%). The dosage may be diluted up to 10 mL.

In some aspects, the subject (e.g., patient) fasts for at least 4 hours, for example 4 to 6 hours, prior to administration of [¹⁸F]-FACBC. For example, administration instructions may advise the subject to fast for at least 4 hours prior to administration of [¹⁸F]-FACBC, and/or a medical professional may advise the subject to fast for at least 4 hours prior to administration of [¹⁸F]-FACBC. The term “fast” means to consume no food or calorie-containing drink. For example, only water (e.g., clear water) or other non-calorie containing fluid, or medications (e.g., prescribed medications) may be consumed within the 4 hours prior to administration.

Additionally or alternatively, the subject may avoid significant exercise for at least one day prior to the PET scan.

The PET imaging technique may utilise scanning devices that detect the 511 keV annihilation photons that are emitted after radioactive decay of fluorine-18. In addition, “micro-PET” scanners that have high spatial resolution can be used for imaging of small animals. In addition to PET scanners, ¹⁸F-radioactivity can also be monitored using one or more radiation detector probes.

In step b) of an exemplary method herein, the acquisition/scanning time can start 3 to 5 minutes after the end of the injection, for example 4 minutes after the end of the injection.

In some examples, the PET scan starts from the subject's vertex (the top of the subject's head) and proceeds to the lowest extremity of the subject (the base of the subject's feet). Thus, for example, the PET scan may include substantially all of the subject's body.

In another aspect there is provided a method of imaging myeloma in a subject, comprising the steps of:
a) administering to the subject a first dose of a detectable amount of [¹⁸F]-FACBC and allowing time for the [¹⁸F]-FACBC to accumulate at one or more areas of interest within the subject;
b) obtaining a first PET scan image of the subject for one or more areas of interest of the subject that include a potential myeloma bone lesion;
c) administering to the subject a second dose of a detectable amount of [¹⁸F]-FACBC and allowing time for [¹⁸F]-FACBC to accumulate at the one or more areas of interest within the subject;
d) obtaining a second PET scan image of the subject of the one or more areas of interest;
e) comparing [¹⁸F]-FACBC uptake in a potential lesion against [¹⁸F]-FACBC uptake in bone marrow of the one or more areas of interest for the first and second PET scan images; wherein the first and second scan images indicate the location and intensity of [¹⁸F]-FACBC and wherein localisation of [¹⁸F]-FACBC indicates the presence of lesion tissue in the subject.

In a further aspect there is provided a method of diagnosing or monitoring myeloma in a subject, comprising the steps of:
a) administering to the subject a first dose of a detectable amount of [¹⁸F]-FACBC and allowing time for the [¹⁸F]-FACBC to accumulate at one or more areas of interest within the subject that include a potential myeloma bone lesion;
b) obtaining a first PET scan image of the subject, wherein the scan image indicates the location and intensity of [¹⁸F]-FACBC;
c) administering to the subject a second dose of a detectable amount of [¹⁸F]-FACBC and allowing time for [¹⁸F]-FACBC to accumulate at the one or more areas of interest within the subject;
d) obtaining a second PET scan image of the subject, wherein the scan image indicates the location and intensity of [¹⁸F]-FACBC;
e) comparing the first and second scan images to determine whether the location and/or intensity of [¹⁸F]-FACBC has increased, remained constant, or decreased.

In at least one embodiment of the second or third aspect, both PET and magnetic resonance imaging (MRI) or X-ray computed tomography (CT) scan images are acquired in steps b) and d), for example, by use of a combined PET-MRI or PET-CT system.

In at least one embodiment, [¹⁸F]-FACBC is administered according to the aspects of the present disclosure discussed above.

The time sufficient for [¹⁸F]-FACBC to accumulate in lesion cells in steps a) and c) of the aspects described above is about 5 minutes or less after [¹⁸F]-FACBC is administered. In some examples, the time taken for [¹⁸F]-FACBC to accumulate is 3 to 5 minutes, e.g., about 4 minutes. This therefore allows image acquisition to start 3 to 5 minutes after administration, e.g., about 4 minutes after [¹⁸F]-FACBC administration.

Once data has been collected from the first PET scan, the images can be visualised and used to view the level, volume and/or location of lesion [¹⁸F]-FACBC uptake within the subject. Images are usually visually interpreted by a nuclear medicine physician or radiologist and standardised uptake values (SUVs) such as SUVmax and SUVmean may be determined.

The time between acquisition of the first and second PET scan images, i.e. between steps b) and d) of the aspects described above, can be as much as one year. In some instances, the time between the first and second PET scans is about 1 month to about 12 months, e.g., a time of 6 months, 5 months, 4 months, 3 months, 2 months, or 1 month or even less than about 1 month. It will be appreciated that steps c) and d) of the second and third aspects described above can be repeated as many times as necessary in order to obtain multiple scan images which can be used to map the development of a lesion over time.

Once image data has been collected from the second PET scan, the first and second images can be visualised together and used to view the change in extent and location of lesion [¹⁸F]-FACBC uptake within the subject, allowing for the diagnosis or monitoring of myeloma. For example, if the level of lesion [¹⁸F]-FACBC uptake has changed then the subject may be diagnosed with myeloma recurrence. In some embodiments, the second scan image can be compared to images of data collected from an earlier PET scan taken before the first PET scan, in addition to comparison with the first PET scan. In addition, any subsequent PET scan images obtained after the second PET scan image can be compared with the first and/or second PET scan images.

By comparing the images from two or more differing time points, the differences in the lesion uptake of [¹⁸F]-FACBC can be analysed. Comparisons can involve qualitative image comparison (e.g. level of lesion uptake to background) and/or quantitative indices derived from the imaging or external radiation detection data (e.g. SUVs). The development, progression or reduction of any lesions can therefore be monitored and diagnosed accordingly. Suitable treatment can then be determined, for example, targeted administration of localised treatment at the site of the lesion. It will be appreciated that the methods described herein can also be used to monitor response to various therapeutic regimens.

The PET scan image obtained in steps b) and d) of the methods described above may be combined with, preceded or followed by anatomical imaging selected from computed tomography (CT) imaging, computerized axial tomography (CAT) imaging or MRI. For combined imaging, the images can be acquired using a dedicated PET-CT, PET-MRI, or separate PET and CT/MRI scanning devices. If separate PET and CT/MRI imaging devices are used, image analysis techniques can be employed to spatially register the PET images with the anatomical images.

In at least one embodiment, the image acquisition steps b) and d) involve obtaining a combined PET scan image and MRI scan image. The PET-MRI images can be obtained using a dedicated PET-MRI scanning device. Such scanning devices are available from Siemens (Biograph mMR) and GE (SIGNA PET/MR). Since MRI does not use ionizing radiation, its use is may be favoured in preference to CT. An advantage of PET-MRI acquisition is that the patient and medical staff only need to be present for a single scan, resulting in a more time and cost efficient process.

In another embodiment, the image acquisition steps b) and d) involve obtaining a combined PET scan image and CT scan image. The PET-CT images can be obtained using a dedicated PET-CT scanning device. An advantage of PET-CT acquisition is that the patient and medical staff only need to be present for a single scan resulting in a more time and cost efficient process.

The methods described herein are intended to be suitable for detecting myeloma bone lesions.

The methods described herein may be suitable for detecting recurrence of myeloma bone lesions.

In an exemplary embodiment, the methods of the present disclosure are used to diagnose recurrent myeloma bone lesions.

The methods herein may include prescribing a treatment regime such as, e.g., medication and/or surgical intervention, among other treatment options, based on analysis of the PET scan image(s). The analysis may include a determination of whether [¹⁸F]-FACBC uptake by a potential myeloma bone lesion is indicative of the presence of myeloma in a subject.

The methods of the present disclosure have use in humans and some methods may have use in non-human animals (for example, dogs and cats).

Another aspect of the present disclosure provides a kit for imaging, diagnosing, and/or monitoring myeloma, comprising: a) [¹⁸F]-FACBC tracer; b) administration instructions in accordance with the aspects of the present disclosure discussed above.

Although the present disclosure has been described above with reference to specific embodiments, it is not intended to be limited to the specific form set forth herein. Rather, the invention is limited only by the accompanying claims and, other embodiments than the specific above are equally possible within the scope of these appended claims.

1. A method of using [¹⁸F]-FACBC, comprising the steps of:
a) administering a detectable amount of [¹⁸F]-FACBC to a subject;
b) acquiring one or more positron emission tomography (PET) scan images of the subject for a region of the subject that includes a potential myeloma bone lesion;
c) comparing ^{[18F]}_{—} FACBC uptake in the potential myeloma bone lesion against [¹⁸F]-FACBC uptake in surrounding bone marrow of the region; and
d) determining whether the potential lesion is a myeloma bone lesion based on the analysis of step c).

2. The method of claim 1, wherein [¹⁸F]-FACBC uptake by the potential lesion is higher than the surrounding bone marrow [¹⁸F]-FACBC uptake.

3. The method of claim 1, wherein step c) includes determining an absence of myeloma bone lesions.

4. The method of claim 1, wherein the dosage of [¹⁸F]-FACBC administered to the subject is 370 MBq (+/−20%).

5. The method of claim 1, wherein the dosage of [¹⁸F]-FACBC administered to the subject is 370 MBq (+/−10%).

6. The method of claim 1, wherein the dosage of [¹⁸F]-FACBC administered to the subject is 370 MBq (+/−5%).

7. The method of claim 1, wherein the dosage of [¹⁸F]-FACBC administered to the subject is 370 MBq.

8. The method of claim 1, wherein the [¹⁸F]-FACBC is injected into the subject as an intravenous bolus.

9. The method of claim 1, wherein [¹⁸F]-FACBC administration is followed by an intravenous saline flush.

10. The method of claim 1, wherein the subject is positioned in a supine position with arms by the side of the subject before the PET scan begins.

11. The method of claim 1, wherein the PET scan starts from the subject's vertex and proceeds to the lowest extremity of the subject.

12. The method of claim 1, wherein the PET scan begins 3 to 5 minutes after the [¹⁸F]-FACBC administration and continues for a total scan time of up to 30 minutes.

13. The method of claim 1, wherein the PET scan starts 4 minutes after the [¹⁸F]-FACBC administration.

14. The method of claim 1, wherein the subject has:
i) consumed no food or calorie-containing drink for at least 4 hours prior to administration of the [¹⁸F]-FACBC, with the exception of clear water and medications; and
ii) avoided significant exercise for at least one day prior to the PET scan.

15. The method of claim 1, comprising:
a) administering a dose of 370 MBq (+/−20%) of [¹⁸F]-FACBC to the subject as an intravenous bolus injection, followed by an intravenous saline flush;
b) acquiring the one or more PET scan images of the subject; and
c) comparing [¹⁸F]-FACBC uptake in the potential myeloma bone lesion against [¹⁸F]-FACBC uptake in the surrounding bone marrow;
wherein the subject is positioned in a supine position with arms by the side before the PET scan begins, the scan starts from the subject's vertex and proceeds to the lowest extremity of the subject, and the scan begins 3 to 5 minutes after the [¹⁸F]-FACBC administration and continues for a total scan time of up to 30 minutes;
wherein the subject has:
i) consumed no food or calorie-containing drink for at least 4 hours prior to administration of the [¹⁸F]-FACBC, with the exception of clear water and medications; and
ii) avoided significant exercise for at least one day prior to the PET scan; and
wherein [¹⁸F]-FACBC uptake by the potential myeloma bone lesion higher than the surrounding bone marrow [¹⁸F]-FACBC uptake is indicative of the presence of myeloma.

16. The method of claim 1, wherein PET and magnetic resonance imaging (MRI) scan images or PET and X-ray computed tomography (CT) scan images are acquired in step b).

17. The method of claim 1, wherein the one or more PET scan images are first one or more PET scan images, the method further comprising:
e) repeating steps a) to d) after a period of time, and comparing the one or more later PET scan images with the first one or more PET scan images to determine whether [¹⁸F]-FACBC uptake by the potential myeloma bone lesion has increased, remained constant, or decreased.

18. The method of claim 17, wherein the period of time is at least 6 months.

19. A method of using [¹⁸F]-FACBC, comprising the steps of:
a) administering a detectable amount of [¹⁸F]-FACBC to a subject; and
b) acquiring one or more positron emission tomography (PET) scan images of the subject for a region of the subject that includes a potential myeloma bone lesion;
wherein [¹⁸F]-FACBC uptake in the potential myeloma bone lesion is compared against [¹⁸F]-FACBC uptake in the surrounding bone marrow to determine whether the potential lesion is a myeloma bone lesion;
wherein the subject is positioned in a supine position with arms by the side before the PET scan begins, the scan starts from the subject's vertex and proceeds to the lowest extremity of the subject, and the scan begins 3 to 5 minutes after the [¹⁸F]-FACBC administration and continues for a total scan time of up to 30 minutes;
wherein the subject has been advised to:
i) consume no food or calorie-containing drink for at least 4 hours prior to administration of the [¹⁸F]-FACBC, with the exception of clear water and medications; and
ii) avoid significant exercise for at least one day prior to the PET scan; and
wherein [¹⁸F]-FACBC uptake by the potential myeloma bone lesion higher than the surrounding bone marrow [¹⁸F]-FACBC uptake is indicative of the presence of myeloma.

20. A method of using [¹⁸F]-FACBC, comprising the steps of:
obtaining one or more positron emission tomography (PET) scan images of a region of a subject that includes a potential myeloma bone lesion, wherein the one or more PET scan images are acquired while the subject is positioned in a supine position with arms by the side, the scan starts from the subject's vertex and proceeds to the lowest extremity of the subject, and the scan begins 3 to 5 minutes after administration of [¹⁸F]-FACBC to the subject and continues for a total scan time of up to 30 minutes;
analysing the one or more PET scan images by comparing [¹⁸F]-FACBC uptake in the potential myeloma bone lesion against [¹⁸F]-FACBC uptake in the surrounding bone marrow; and
determining a status of the potential myeloma bone lesion based on the analysis, wherein [¹⁸F]-FACBC uptake by the potential myeloma bone lesion higher than the surrounding bone marrow [¹⁸F]-FACBC uptake is indicative of the presence of myeloma.

21. The method of claim 20, further comprising prescribing a treatment regime for the subject based on the determined status of the potential myeloma bone lesion.
